(19) United States
(12) Patent Application Publication
(10) Pub. No.: US 2003/0191361 A1
(43) Pub. Date: Oct. 9, 2003

(54) Boxer-style undergarment with integrated erection-control device

(76) Inventor: David Luis Flores, Vista, CA (US)

Correspondence Address: David Flores, 730 Breeze Hill Road #276, Vista, CA, 92083, US

(21) Appl. No.: 10/116,987

(22) Filed: Apr. 8, 2002

(51) Int. Cl. A61F 5/00

(52) U.S. Cl. 600/38

(57) ABSTRACT

A penis/genital pouch and two connected leg straps are integrated into a single waistband shared by that of the same of a boxer style undergarment. A male's penis and genitalia rest in the pouch. The two leg straps, elastic in nature, restrict erections by fitting snugly under the scrotum and around the lower buttocks. This gives the overall appearance, breathe ability, and comfort of boxer style shorts with the support and ease of mind of underwear.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT

### DISK APPENDIX

Not Applicable

### BACKGROUND OF THE INVENTION

This invention relates to the problems of discomfort and visible erections caused by modem day undergarments in the field of clothing.

For many years, males have been wearing different types of undergarments in addition to normal day wear (pants and shorts). The primary undergarments at the time (prior art) are underwear, boxer shorts, and briefs. With a variety of different choices, it is needless to say that each option possesses its own disadvantages.

Classic underwear (U.S. Pat. No. 190 6,353,940) provides descent support for the genitals, and at the same time offers reasonable concealment of erections. However, because underwear covers a large portion of a male's posterior, it can position itself between a person's buttocks and cause great discomfort. The excessive and unnecessary amount of material which is in constant contact with the skin also causes trapped heat, especially while engaged in physical activity, leading to awkward sweat locations. Additionally, underwear does not provide the desired style which boxer shorts offer.

Boxer shorts (U.S. Pat. No. D382,386) are very desirable among men because of the style which they provide. However, they offer no restrictions in case of an erection. This causes an embarrassing situation which one hopes to avoid altogether. Furthermore, they offer no support while engaging in physical activities.

The undergarment known as briefs (U.S. Pat. No. D445,239) lack the style of boxer shorts, and cause even greater discomfort than underwear due to the increased amount of trapped heat.

### BRIEF SUMMARY OF THE INVENTION

A support apparatus consisting of a fabric genital and penis supporter pouch with dual elastic straps is integrated into a boxer shorts-style undergarment, providing superior benefits of comfort, style, and dignity.

The object and advantages of my invention are to provide an undergarment with the style and breathe ability of boxer shorts, as well as possessing the support and restriction which underwear provides. This will enable the user to hide an erection while the device is worn under pants, shorts, or similar articles of clothing. It will also provide ample support of genitalia while engaged in physical activities.

### BRIEF DESCRIPTION OF THE SEVERAL VIEWS OF THE DRAWING

FIG. 1 depicts a side view of the invention with emphasis on the inner support device FIG. 2 shows the same side view as FIG. 1, but with an emphasis on the outer portion of the invention. FIG. 3 portrays a frontal view of the invention with an emphasis on the inner support device. FIG. 4 shows the same frontal view with an emphasis on the outer portion of the invention. FIG. 5 shows a side view of the invention being worn by a male. FIG. 6 shows a frontal view of the device while being worn by a male.

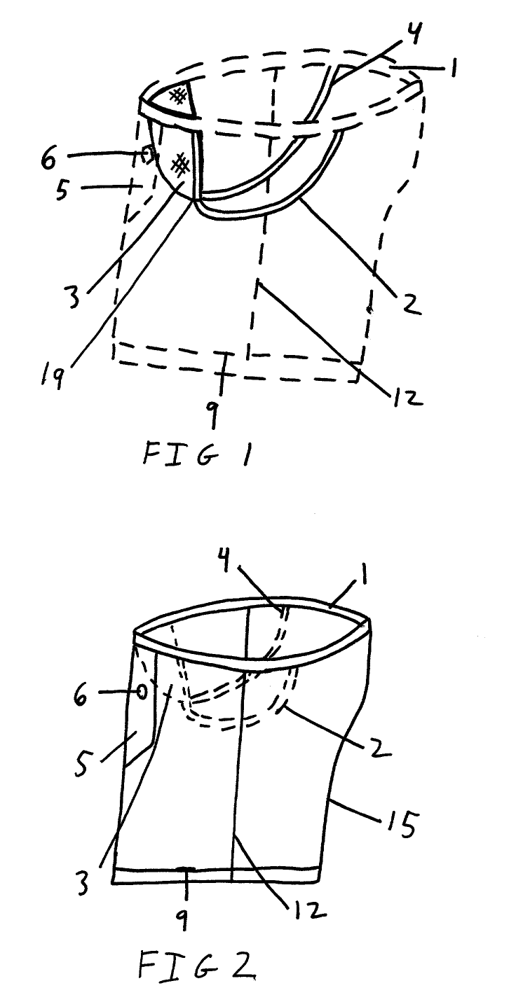

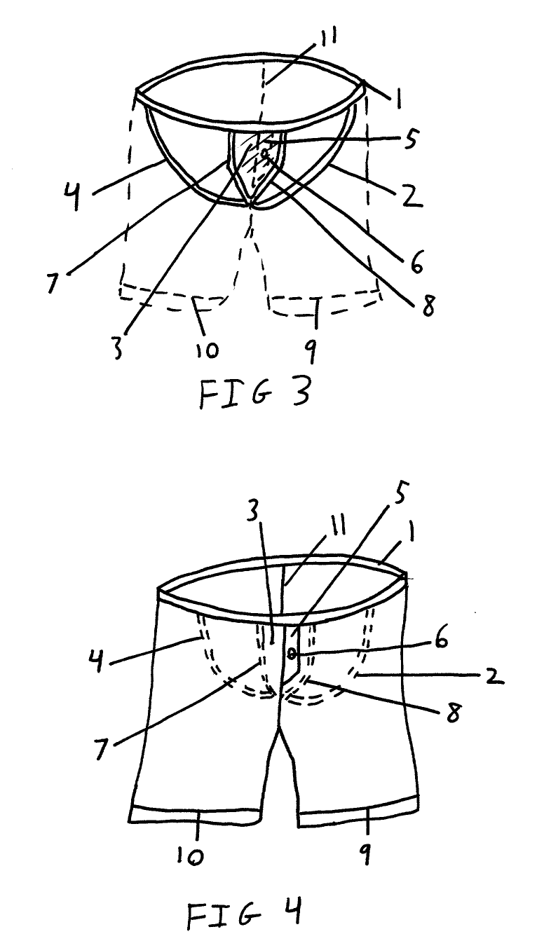

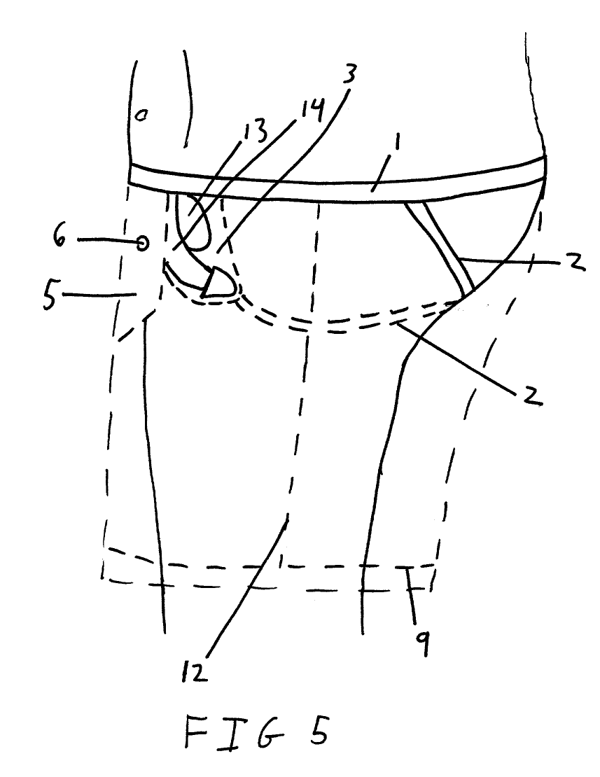

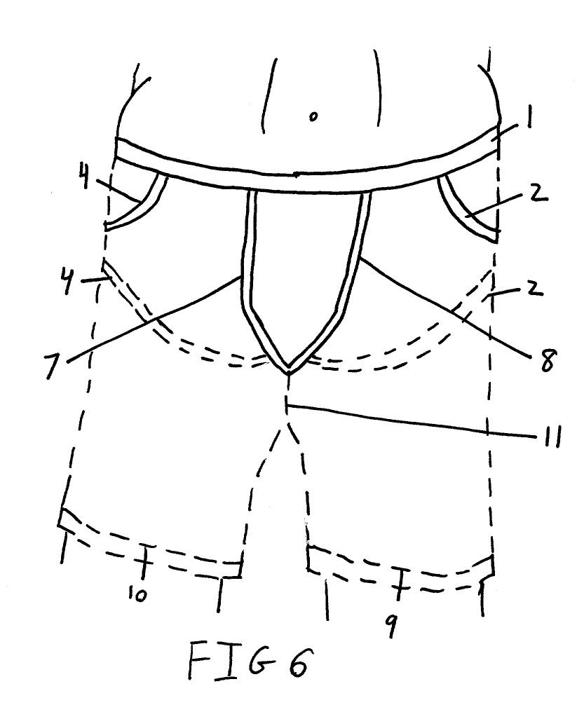

### DETAILED DESCRIPTION OF THE INVENTION

In the invention, the upper edge of the penis/genital pouch 3, with sides 7,8, is bounded to the elastic waistband 1. Two leg straps 2,4 of elastic quality are connected to the lower tip 19 of the penis/genital pouch 3 and at separate positions on the waistband 1 of approximately equal distance from the penis/genital pouch 3. Boxer-style undergarment shorts 15 are also fabricated on the same waistband 1.

The premiere material for the penis/genital pouch 3 is said to be cotton, silk, or that which does not cause irritation to the skin. This pouch 3 will have sides 7,8 sewn of slightly elastic or restrictive material so the genitalia do not accidentally position itself outside of the pouch. The top of the said pouch 3 will be sewn to an elastic or neutral waistband 1 and the bottom tip 19 of the pouch 3 will be sewn to two elastic fabric leg straps 2,4. Each of the two remaining ends of the leg straps 2,4 will also be fabricated into the waistband 1. Furthermore, the waistband 1 will also be sewn to boxer-style shorts 15. These boxer-style shorts 15 can consist of many different styles and may include a functional fly 5 with button fastener 6. When wearing the invention, a male's penis 14 and his testicles 13 rest in the concave side of the penis/genital pouch 3. The leg straps 2,4 hug the inside of the legs 17 under the scrotum, pass under the buttocks 16, and return to the waistband 1 at opposite sides rear of the penis/genital pouch 3. The outward appearance of this device is that of a stylish boxer-style undergarment 15. However, the system of an integrated penis/genital pouch 3 and restrictive leg straps 2,4 ensure that others are unaware of the occurrence of an erection, while at the same time provide the solid comfort and feel of wearing only one undergarment.

I claim:

1. A male erection control garment consisting of a penis/genital pouch and two supporting straps integrated into a waistband, where said waistband is also integrated into an undergarment.

2. Erection control garment of claim 1 with waistband integrated into garment of waterproof nature.
